(19) United States
(12) Patent Application Publication
(10) Pub. No.: US 2016/0166360 A1
(43) Pub. Date: Jun. 16, 2016

(54) RESORBABLE EXPANDING MESH IMPLANT PARTIALLY CAPPED WITH A BARRIER MEMBRANE

(71) Applicant: Iwanczyk BARTLOMIEJ, Warszawa (PL)

(72) Inventor: Wanczyk Bartlomiej, Warsaw (PL)

(21) Appl. No.: 14/905,098

(22) PCT Filed: Jul. 25, 2014
(86) PCT No.: PCT/PL2014/050045

§ 371 Date: Jan. 14, 2016

(30) Foreign Application Priority Data
Jul. 25, 2013 (PL) P.404840

(51) Int. Cl. A61C 8/00; A61L 27/24; A61L 27/58

(52) U.S. Cl. CPC A61C 8/0092; A61L 27/58; A61L 27/24; A61C 8/0033; A61C 8/0012; A61L 2430/02; A61L 2430/12

(57) ABSTRACT

A resorbable implant in the shape of a cylinder composed of a resorbable mesh (2) capable of expansion, capped in its upper portion with a barrier membrane (1) with a free edge (3) that makes it possible to dress a sinus-oral connection with simultaneous bone regeneration during extractions, closed-type sinus lifts and accidental sinus mucosal damage during preparation for tooth implantation

The subject of the present invention is a resorbable, expanding mesh implant partially capped with a resorbable barrier membrane. The present invention is designed for medical use in the areas of dental surgery, maxillary surgery and maxillofacial surgery. The present invention can be used in directed bone reconstruction procedures, post-implantation procedures and following tooth extractions.

Many implants that are introduced into organisms are known. Some implants, like stents, undergo expansion. Medical applications also make use of resorption mechanisms. Resorbable materials include highly crosslinked natural collagen membranes as well as synthetic materials such as polylactide, polyglycolide, polylactideglycolide, polycaprolactone, polydioxane, trimethylene carbonate, poly-β-hydroxybutyrate, poly-g-ethylglutamate, poly-DTH-iminocarbonate, poly-biphenyliminocarbonate, poly-orthoester, poly-cyanoacrylate, poly-osphazene).

During the maxillary sinus lift procedure it is known to use biomaterials for controlled bone regeneration as well as barrier membranes composed of highly crosslinked collagen, in order to separate the osteogenesis process from the epithelial proliferation.

During maxillary sinus lifts, using both the closed and open methods, the performation of the mucous membrane is capped off with a barrier membrane composed of highly crosslinked collagen.

The subject of the present invention is the resorbable expanding mesh implant, partially capped with a barrier membrane. The implant is built in its entirety out of resorbable materials. The implant is composed of a cylindrical mesh lattice 2 with rhomboid cells, set out in such a way that their acute angle is directed towards the base of the lattice. This orientation facilitates the expansion and stretching of the lattice. At one end of the cylindrical lattice 2, the implant possesses a hemispherically shaped barrier membrane, particularly composed of a highly crosslinked collagen membrane, that forms the apical dome 1 of the implant. There is a permanent attachment site 4 of the apical dome 1 with the cylindrical mesh lattice 2, wherein a cylindrical lattice 2 and hemispherical dome 1 are attached via stitching with resorbable thread or else a permanent connection such as a thermal or chemical connection. The hemispherical apical dome 1 of the implant is connected such that a free edge 3 remains from about ⅙ to ½, optimally about ⅕ of the way between the edge of the dome and the attachment site 4 of the dome to the lattice, due to which the free edge 3 of the dome forms the implant flange. The implant dome 1 should constitute between ½ and ⅙ of the total length of the implant, optimally ⅓.

The subject of the present invention in its example embodiment is illustrated by the following figures, wherein

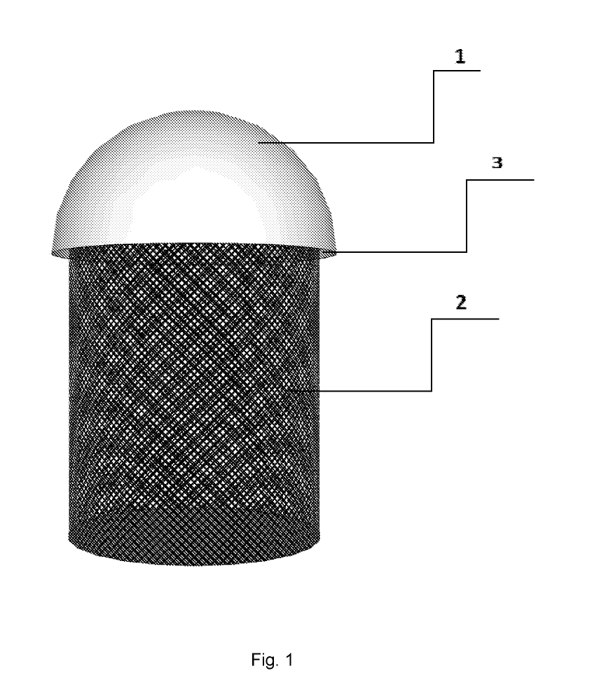

FIG. 1 represents a side view of the implant,

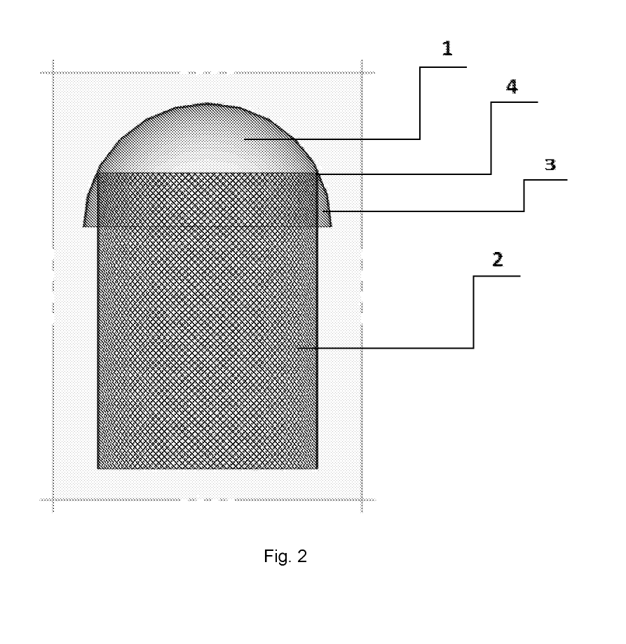

FIG. 2 is a cross-section through the implant and

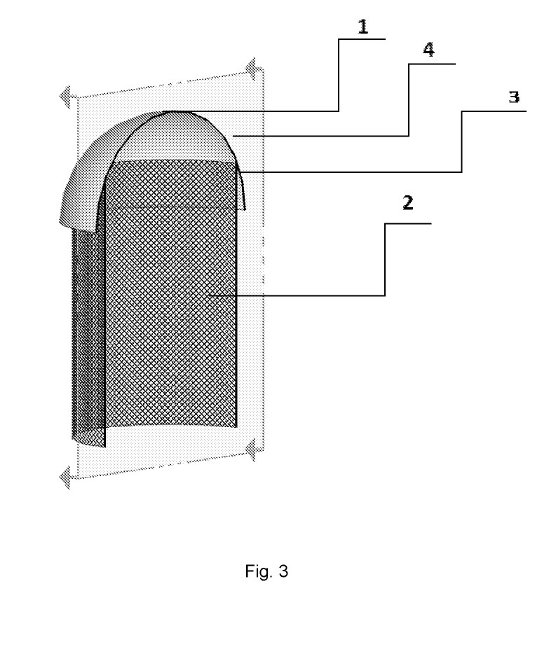

FIG. 3 represents a side view of the implant in a diagonal plane.

According to FIG. 1, FIG. 2 and FIG. 3 the implant consists of:

1 The apical dome composed of a highly crosslinked barrier membrane,

2 A cylindrical mesh lattice made up of rhomboid cells,

3 The free edge of the apical dome constituting the implant flange,

4 The attachment site of the apical dome with the cylindrical mesh lattice.

The principle of the present invention is based on the possibility of introducing the implant into the implantation site, despite the break in the sinus mucous membrane, and to regenerate the bone with out the need to delay the procedure until the perforation of the sinus mucosa is healed.

Due to the use of the flange 3, the structure of the invention makes it possible for the implant to wedge itself underneath the sinus mucous membrane at the edge of the maxillary crestal bone, which constitutes the bottom of the maxillary sinus. Due to this, it is possible to tightly delimit the osteogensis site from the epithelial proliferation, and prevents biomaterial ingress into the maxillary sinus lumen eliminating possible dangerous inflammatory complications. The cylindrical lattice 2 composed of rhomboid cells undergoes expansion and facilitates the sealed and stable condensation of biomaterial for osteoregeneration in lesions of various diameters, such as in post-extraction dental sockets. The use of mesh apertures enables the material to adhere to bone walls, which facilitates osteogenesis at the planned site. Moreover, there is also the possibility of stabilizing the implant through affixing the lattice 2 mesh using pins or bone screws.

1. A resorbable expanding mesh implant partially capped with a barrier membrane, composed of resorbable synthetic or natural materials, characterised in that it is composed of a cylindrical mesh lattice (2) in which the rhomboidal cells are oriented towards the base of the cylinder with the acute angle of the rhombus, and one side of the lattice is equipped with a hemispherically shaped barrier membrane forming the implant dome (1), wherein the implant dome (1) constitutes between ½ and ⅙ of the total length of the implant, as measured from the apex of the apical dome (1) of the implant to the base of the cylindrical lattice (2), and wherein between the hemispherical apical dome of the implant and the edge of the lattice there is a permanent connection (4), wherein the connection (4) is located between the outermost cells of the lattice (2) located in the concave portion of the hemispherical apical dome and the apical dome (1), at from ⅙ to ½ of the height of the apical dome (1) from the edge of the dome, whereas the free edge of the dome constituting from about ⅙ to ½ of the dome height constitutes the free edge (3) which forms the implant flange.

2. Implant according to claim 1, wherein permanent connection (4) consists of a stitch of resorbable suture or another permanent connection such as a chemical or thermal connection.

3. Implant according to claim 1, wherein hemispherically shaped barrier membrane is composed of highly crosslinked collagen.

4. Implant according to claim 1, wherein the implant dome (1) constitutes ⅓ of the total length of the implant.

5. Implant according to claim 1, wherein the connection (4) is located between the outermost cells of the lattice (2) located in the concave portion of the hemispherical apical dome and the apical dome (1), at ⅕ of the height of the apical dome (1) from the edge of the dome.

6. Implant according to claim 1, wherein the free edge of the dome constituting ⅕ of the dome height constitutes the free edge (3), which forms the implant flange.
